(19) United States
(12) Patent Application Publication
(10) Pub. No.: US 2025/0064064 A1
(43) Pub. Date: Feb. 27, 2025

(54) METHODS FOR DESIGNING AND PERFORMING A VAPOR PHASE HYDROGEN PEROXIDE DECONTAMINATION CYCLE

(71) Applicant: Amgen Inc., Thousand Oaks, CA (US)

(72) Inventors: Evan Kruchowy, Port Hueneme, CA (US); Sai Chakradhar Padala, Thousand Oaks, CA (US); Daniel Marsiglio, Los angeles, CA (US)

(21) Appl. No.: 18/719,164

(22) PCT Filed: Dec. 12, 2022
(86) PCT No.: PCT/US2022/052507

§ 371 Date: Jun. 12, 2024

Related U.S. Application Data
(60) Provisional application No. 63/288,758, filed on Dec. 13, 2021.

(51) Int. Cl. A01N 59/00; A01P 1/00; A61L 2/20; A61L 101/02

(52) U.S. Cl. CPC A01N 59/00; A01P 1/00; A61L 2/208; A61L 2101/02; A61L 2202/122

(57) ABSTRACT

A method for decontaminating a chamber interior using a VPHP decontamination cycle includes sealing the chamber interior, circulating a dose of gas in the chamber interior for a dwell time, and aerating the chamber interior until no more than an allowable remaining amount of the gas remains in the chamber interior. A method for selecting parameters for a VPHP decontamination cycle for a chamber interior includes performing a plurality of experiments testing different amounts of gas and different amounts of time, selecting a dose of the gas to be a least amount of gas that maintains full saturation of the chamber interior over a selected time, and selecting a dwell time such that the dwell time is both less than or equal to the selected time and a least amount of time for which, after circulating the dose of the gas, the remaining amount of viable spores is allowable.

### FIELD OF THE DISCLOSURE

The present application relates generally to vapor phase hydrogen peroxide decontamination cycles, and more specifically to the design (e.g., parameter selection) and/or performance of vapor phase hydrogen peroxide decontamination cycles.

### BACKGROUND

Vapor phase hydrogen peroxide (VPHP) decontamination is a method of decontamination that may be used in industries including life sciences, chemical sciences, pharmaceuticals, medical, electrical engineering, manufacturing, assembly, and other applications by circulating a gas comprising hydrogen peroxide in a closed-loop airflow. Applications for VPHP decontamination include decontaminating chamber interiors such as rooms, air locks, clean rooms, isolators, laminar air flow workbenches, biological safety cabinets, restricted-access barrier systems, incubators, and decontamination chambers, for example. Equipment used for performing VPHP decontamination cycles is often standalone equipment. VPHP is commonly used, for example, for its ability to permeate a wide variety of materials, its ability to maintain dryness of the chamber interior being decontaminated and not leave behind residue, its low toxicity, its low operating cost, its ability to reduce cross contamination due to minimal equipment intrusion, and its compatibility with various geometries of chamber interiors.

One specific application of VPHP decontamination is for decontaminating chambers of isolators used to manufacture drug products (e.g., at the “fill” stage in which vials or other containers are filled with a drug product). The FDA, in its Guidance for Industry Sterile Drug Products Produced by Aseptic Processing—Current Good Manufacturing Practice, recommends that colony forming unit (CFU) count be reduced by a minimum of 6-Logs (or reduced to 1 ppm) for isolators. One way of assessing whether a VPHP decontamination cycle achieves the desired CFU reduction is by using biological indicators (Bls). A BI may be manufactured to have a specific D-value. The D-value is the time required to reduce CFUs by 1-Log (or 90%), following the equation: CFU=CFUᵢ*10 −1/Dt, where CFU is a current remaining CFU count, CFUᵢ is an initial CFU count, D is the manufacturer-assigned D-value, and t is exposure time. A D-value is unique to a particular decontamination condition and isolator design, and therefore is not directly translatable to other decontamination conditions and isolators. The BI manufacturer generally provides a D-value for each BI lot that may have been tested using a different isolator than the end-user's isolator. Even if determined using a different isolator, a manufacturers' D-value can still provide some indication of the relative kill difficulty of the BI lot.

Factors that affect the efficacy of a VPHP decontamination cycle at achieving FDA-recommended levels of CFU reduction include: VPHP exposure time (dwell time), VPHP dosage, VPHP circulation, the D-value of a BI, initial CFU count, location of the Bls within the isolator, isolator design, and humidity and temperature within the isolator. Generally, parameters of the VPHP decontamination cycle that can be controlled include VPHP exposure time (dwell time), VPHP dosage, VPHP circulation, and humidity and temperature inside the chamber. In some cases, isolator configuration may also be controllable to some extent.

CFU reduction is not the only consideration in VPHP decontamination. Efficiency of the VPHP decontamination cycle is another important consideration. “Efficiency” of VPHP decontamination may refer to efficiency in time (i.e., how long the VPHP decontamination cycle takes), efficiency of materials (e.g., amount of gas consumed), and/or efficiency of human labor (e.g., number of man hours required to perform the VPHP decontamination cycle), for example.

Efficiency with respect to time affects throughput of an isolator, as a lower time-efficiency of the VPHP decontamination cycle will result in more downtime for the isolator, and thus reduce the filling rate for the isolator. It is worth noting, in many applications, a large isolator capable of high-throughput filling is not feasible for a variety of reasons, including cost, transportation logistics, physical space, etc. Therefore, small isolators with lower throughputs than larger isolators are often used. For these small isolators, it may be even more important that the throughput not be further reduced by a lengthy and inefficient decontamination cycle. Thus, careful determination of parameters for a VPHP decontamination cycle can be critical.

Conventional VPHP decontamination may determine parameters for a VPHP decontamination cycle using an “overkill” approach, such as using a longer dwell time and larger dosage than is necessary to achieve desired CFU reduction. However, this “overkill” approach presents certain disadvantages. For example, while a dose of gas that is too small may not achieve the desired CFU reduction, a dose of gas that is too large may both reduce efficiency (e.g., with respect to time, materials, and possibly human labor) and create condensation on surfaces of the chamber interior. Furthermore, while too short of a dwell time may not achieve desired CFU reduction, too long of a dwell time may reduce efficiency (e.g., with respect to time and possibly human labor). The “overkill” approach also requires guesswork (e.g., trial by error), and is therefore inconsistent, dependent on the operator, prone to errors, and inefficient (e.g., with respect to time, materials, and possibly human labor).

### BRIEF SUMMARY

One aspect of the present disclosure provides a method for decontaminating a chamber interior using a vapor phase hydrogen peroxide decontamination cycle, the method including (a) sealing the chamber interior from an outside environment while a population of viable spores is in the chamber interior; (b) circulating a dose of gas comprising hydrogen peroxide in the chamber interior for a dwell time, wherein: the dose of the gas is, within a first tolerance range, a least amount of the gas that, when circulated in the chamber interior, maintains full saturation of the chamber interior over the dwell time, and the dwell time is, within a second tolerance range, a least amount of time in which the population of viable spores is exposed to the dose of the gas to reduce the population of viable spores to an allowable remaining amount; and (c) after circulating the dose of the gas in the chamber interior for the dwell time, aerating the chamber interior until no more than an allowable remaining amount of the gas remains in the chamber interior, wherein the first tolerance range and the second tolerance range are each no more than 20%.

Another aspect of the present disclosure provides a method for selecting a dose and a dwell time for a decontamination cycle for a chamber interior using a vapor phase hydrogen peroxide decontamination cycle, the method including: (a) performing a plurality of experiments testing a plurality of amounts of gas comprising hydrogen peroxide and a plurality of amounts of time, wherein performing the plurality of experiments includes, for each experiment of the plurality of experiments: circulating a an amount of gas of the plurality of amounts of gas in the chamber interior for an amount of time of the plurality of amounts of time, monitoring concentration of the amount of gas in the chamber interior over the amount of time, and after circulating the amount of gas in the chamber interior for the amount of time, determining a remaining amount of viable spores in the chamber interior; (b) selecting the dose of the gas to be a least amount of gas, from among the plurality of amounts of gas, that maintained full saturation of the chamber interior over a selected time when circulated in the chamber interior during the plurality of experiments; and (c) selecting the dwell time such that the dwell time is: (i) less than or equal to the selected time, and (ii) a least amount of the plurality of amounts of time for which, when the dose of the gas was circulated in the chamber interior for the dwell time during the plurality of experiments, the determined remaining amount of viable spores did not exceed an allowable remaining amount.

### BRIEF DESCRIPTION OF THE DRAWINGS

The skilled artisan will understand that the figures described herein are included for purposes of illustration and are not limiting on the present disclosure. The drawings are not necessarily to scale, emphasis instead being placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 depicts an example chamber interior of an isolator.

FIG. 2 is a graph depicting example hypothetical experimental results for VPHP concentration over time for various doses of gas comprising hydrogen peroxide.

FIG. 3 is a table depicting example hypothetical experimental growth results for a plurality of biological indicators after exposure to different doses of gas for different dwell times.

FIG. 4 is a flow diagram depicting an example method of decontaminating a chamber interior using a vapor phase hydrogen peroxide decontamination cycle.

FIG. 5 is a flow diagram depicting an example method of selecting parameters for a decontamination cycle for a chamber interior using a vapor phase hydrogen peroxide decontamination cycle.

### DETAILED DESCRIPTION

The present disclosure aims to reduce problems with conventional approaches (e.g., as described in the Background section) by providing improved method(s) for designing and/or performing a VPHP decontamination cycle. The method(s) may include determining a dwell time and a dose which are each large enough to achieve desired CFU reduction, without the inefficiencies of conventional VPHP decontamination and without causing humidity increase and condensation, and, for the example of an isolator, possibly harming quality of the product.

Furthermore, in some embodiments, the disclosed method(s) may be partially or entirely automated, thereby not only improving efficiency with respect to human labor, but also removing inconsistencies due to reliance on operator technique.

The various concepts introduced above and discussed in greater detail below may be implemented in any of numerous ways, and the described concepts are not limited to any particular manner of implementation. Examples of implementations are provided below for illustrative purposes.

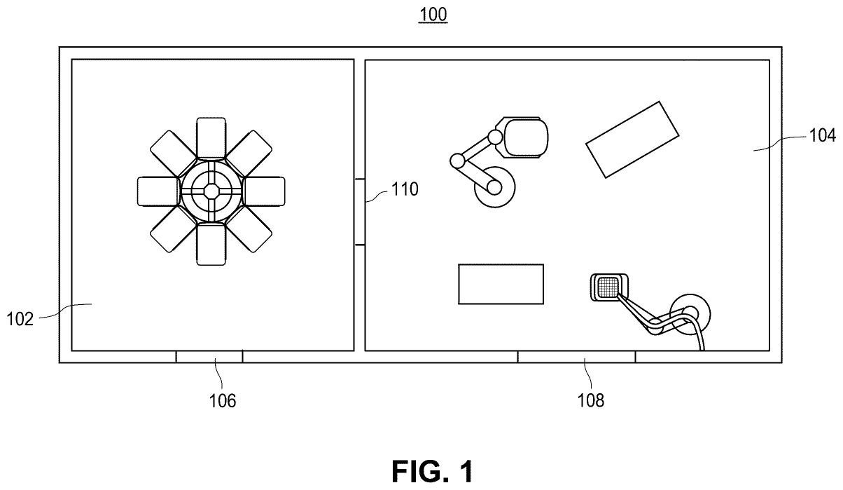

FIG. 1 depicts a top-down view of an example chamber interior of an isolator 100 that may be decontaminated in accordance with the VPHP decontamination techniques described herein. The isolator illustrated in FIG. 1 may be standalone equipment, such as the Vanrx® SA 25 filling machine, though in other examples the isolator 100 may be incorporated into other equipment. The isolator 100 may be used for filling containers, including vials, syringes, cartridges, tubes, beakers, cups, or any other suitable holding structure, with liquids, solids, gases, or plasmas (e.g., drug products). The isolator 100 may be a gloveless isolator, and/or the isolator 100 may use peristaltic filling.

In this example, the isolator 100 includes two chambers: a decontamination staging isolator (DSI) chamber 102 and a filling isolator chamber 104. The DSI chamber 102 may be a staging area for the containers and the filling isolator chamber 104 may be an area for filling the containers. The DSI chamber 102 may contain equipment for holding, staging, moving, or storing containers, such as a carousel. The filling isolator chamber 104 may contain equipment for filling containers, such as filling nozzles. In the example shown, the interior volume of the filling isolator chamber 104 is larger than the interior volume of the DSI chamber 102.

The DSI chamber 102 and the filling isolator chamber 104 have exterior doors 106 and 108, respectively. Each of the exterior doors 106 and 108 can be individually opened to access each of the DSI chamber 102 and the filling isolator chamber 104. Between the DSI chamber 102 and the filling isolator chamber 104, is an interior door 110 which can be opened to allow for container transfer between the DSI chamber 102 and the filling isolator chamber 104.

In one example, if one of the exterior doors 106 or 108 is opened and the interior door 110 is closed, then the chamber (either the DSI chamber 102 or the filling isolator chamber 104) with the open exterior door may be considered contaminated. In another example, if the interior door 110 is open when one of the exterior doors 106 or 108 is opened, then both the DSI chamber 102 and the filling isolator chamber 104 may be considered contaminated. In another example, if both exterior doors 106 and 108 are closed and one of the chambers (either the DSI chamber 102 or the filling isolator chamber 104) is considered contaminated, and the interior door 110 is opened, both the DSI chamber 102 and the filling isolator chamber 104 may then be considered contaminated. In another example, if both exterior doors 106 and 108 are open, regardless of the state of the interior door 110, both the DSI chamber 102 and the filling isolator chamber 104 may be considered contaminated.

Decontaminating all of, or a portion of, the isolator 100 may be accomplished by any number of decontamination cycles. Three example types of decontamination cycles, (i) Full Cycle Decontamination, (ii) Filling Isolator Decontamination Cycle, and (iii) Decontamination Cycle Isolator Decontamination Cycle, are described herein.

The Full Cycle Decontamination may be performed with the interior door 110 open, both the exterior doors 106 and 108 closed, and gas including hydrogen peroxide being circulated in both the DSI chamber 102 and the filling isolator chamber 104. Assuming that prior to the Full Cycle Decontamination, both the DSI chamber 102 and the filling isolator chamber 104 were considered contaminated, after performing the Full Cycle Decontamination, the filling isolator chamber 104 may be considered decontaminated and the DSI chamber 102 may be considered contaminated. After performing the Full Cycle Decontamination, seals on the interior door 110 may also be considered decontaminated.

The Filling Isolator Decontamination Cycle may be performed with the interior door 110 closed, the exterior door 108 of the filling isolator chamber 104 closed, and gas including hydrogen peroxide being circulated in the filling isolator chamber 104. After performing the Filling Isolator Decontamination Cycle, the filling isolator chamber 104 may be considered decontaminated.

The Decontamination Staging Isolator Decontamination Cycle may be performed with the interior door 110 closed, the exterior door 106 of the DSI chamber 102 closed, and gas including hydrogen peroxide being circulated in the DSI chamber 102. After performing the Decontamination Staging Isolator Decontamination Cycle, the DSI chamber 102 may be considered decontaminated.

In general, performing one of the three decontamination cycles listed above includes circulating a dose of the gas including hydrogen peroxide into the isolator chamber or chambers of interest, waiting for a dwell time to elapse, then aerating and removing the gas until the residual gas concentration level is less than some threshold amount (e.g., 1 ppm). The isolator 100 may have multiple chambers being decontaminated. As used herein, the term “chamber interior” means all of the (one or more) chambers in which gas comprising hydrogen peroxide is circulated for the purpose of a VPHP decontamination cycle. For example, in the Full Cycle Decontamination, the gas is circulated in both the filling isolator chamber 104 and the DSI chamber 102; therefore, in this example, the aggregate of the interiors of these two chambers would be referred to as the “chamber interior.” In another example, in the Filling Isolator Decontamination Cycle, the gas is only circulated in the filling isolator chamber 104; therefore, in this example, only the interior of the filling isolator chamber 104 would be referred to as the “chamber interior.”

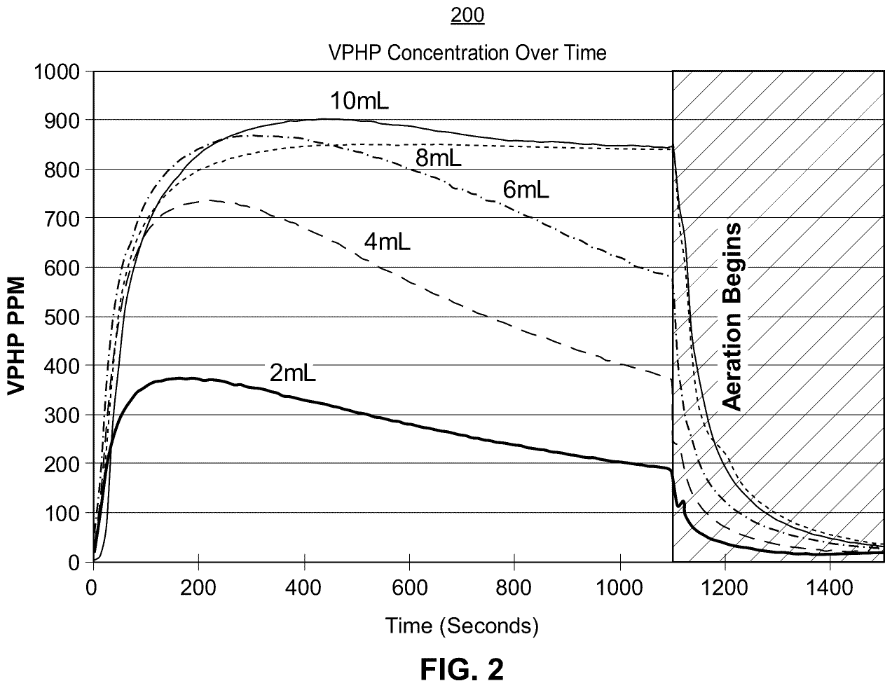

FIG. 2 depicts a graph 200 depicting example hypothetical experimental results for VPHP concentration over time for various doses of a gas comprising hydrogen peroxide. Generally, the example experimental results included in FIG. 2 may be used in determining dose for an example VPHP decontamination cycle.

The example hypothetical experimental data included in FIG. 2 may correspond to a plurality of experiments with varied amounts of a gas that includes hydrogen peroxide. FIG. 2 depicts hypothetical results of an experiment including testing five amounts of the gas (specifically, 10 mL, 8 mL, 6 mL, 4 mL, and 2 mL). It is noted the gas may include different concentrations of hydrogen peroxide. For example, the gas may by 10% hydrogen peroxide, 20% hydrogen peroxide, 30% hydrogen peroxide, 40% hydrogen peroxide, 50% hydrogen peroxide, 60% hydrogen peroxide, 70% hydrogen peroxide, 80% hydrogen peroxide, or any other suitable concentration of hydrogen peroxide.

In FIG. 2, parts per million (ppm) of VPHP for the five amounts of the gas are plotted as a function of time. There are three main intervals of time depicted in FIG. 2. In chronological order, they are: (i) an introduction phase in which the gas is introduced to the chamber interior, (ii) a circulation phase in which the gas circulates throughout the chamber interior, and (iii) an aeration phase in which the gas is removed from the chamber interior.

Turning first to the introduction phase, towards the beginning of the recorded time span (roughly the first 200 seconds, in this example), the five curves each rise steeply from 0 ppm up to a higher ppm, corresponding to the gas being introduced into the chamber interior. The introduction phase may be considered complete once the full amount of gas is introduced to the chamber interior.

Turning next to the circulation phase (starting at roughly 200 seconds and ending at roughly 1100 seconds, in this example), the gas circulates through the chamber interior. The 2 mL, 4 mL, and 6 mL amounts of gas each decline in VPHP ppm during the circulation time interval. However, the 8 mL and 10 mL amounts of gas each show a plateau or “table top” behavior over the circulation time interval. This table top behavior suggests that the 8 mL and 10 mL amounts of the gas are each sufficient to maintain full saturation of the chamber interior over the circulation time interval. Full saturation of the chamber interior corresponds to the maximum VPHP ppm that can be present inside the chamber interior. Further introduction of the gas above the full saturation point does not increase VPHP ppm inside the chamber interior, and instead may cause undesired condensation of the gas on surfaces of the chamber interior.

Finally, turning to the aeration phase (starting at roughly 1100 seconds, in this example), the five curves each fall steeply, reflecting the removal of gas from the chamber interior. The aeration phase may be considered complete once the VPHP ppm falls below a threshold (e.g., 1 ppm). It is worth noting that the larger the amount of the gas for each of the five amounts of the gas, the longer it takes for VPHP ppm to fall below the desired threshold.

As noted above, based on the example hypothetical results illustrated in FIG. 2, both the 8 mL amount of the gas and the 10 mL amount of the gas are sufficient to maintain full saturation of the chamber interior over the circulation time interval. However, the 10 mL amount of the gas is more likely to cause condensation (or more condensation) on surfaces of the chamber interior than the 8 mL amount of the gas. Additionally, as seen in FIG. 2, the 8 mL amount of gas is aerated and removed from the chamber interior more quickly than the 10 mL amount of gas. For at least these reasons, in this example, 8 mL is the preferred amount of gas out of those tested in the five experiments. Accordingly, 8 mL may be selected as the dose, in this example.

The result of 8 mL as the dose may be unique to this example and may depend, for example, on the chamber interior being decontaminated. For example, if the chamber interior is the isolator 100 illustrated in FIG. 1, the dose may vary between the three different described decontamination cycles. Furthering this example, the Full Cycle Decontamination may have a larger chamber interior volume than the Decontamination Staging Isolator Decontamination Cycle. Accordingly, the dose required for the Full Cycle Decontamination may be larger than the dose required for the Decontamination Staging Isolator Decontamination Cycle. It may also be the case that the difference in chamber volume interior between the Full Cycle Decontamination and the Filling Isolator Decontamination Cycle is not significant enough to warrant different doses. Accordingly, the dose required for the Full Cycle Decontamination may be the same as the dose required for the Filling Isolator Decontamination Cycle.

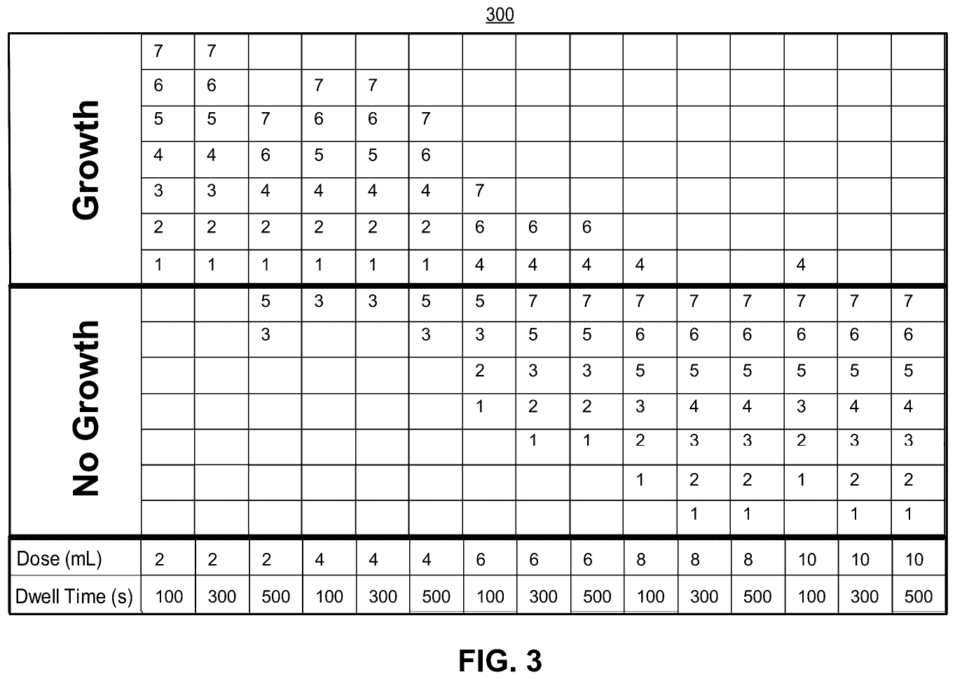

FIG. 3 depicts a table 300 of experimental hypothetical growth results for biological indicators after exposure to different amounts of gas for different amounts of times in the chamber interior. Generally, the example experimental results included in FIG. 3 may be used in determining dwell time for a VPHP decontamination cycle.

In FIG. 3, example hypothetical data are depicted for a plurality of experiments. The hypothetical data depicted in FIG. 3 may correspond to the same or different experiments as the hypothetical data depicted in FIG. 2. Regardless of whether the hypothetical data depicted in FIG. 3 corresponds to the same experiments as the hypothetical data depicted in FIG. 2, the hypothetical data depicted in FIG. 3 may correspond to the same isolator as the hypothetical data depicted in FIG. 2, or a different isolator, either or both of which may be the isolator 100 of FIG. 1.

In general, for each of the experiments, a respective amount of gas is circulated in a chamber interior containing Bls (in these experiments, seven Bls) for a respective amount of time. Then, after removing the gas from the chamber interior, the Bls are tested to determine whether the desired CFU reduction was achieved.

One example method of determining whether the desired CFU reduction was achieved is by removing the Bls from the chamber interior after circulating a particular amount of gas for a particular amount of time, and incubating the Bls in a growth-promoting medium for seven days. Assuming the Bls were inoculated with more than one million CFUs and assuming the desired CFU reduction is 6-Log reduction, no growth after seven days indicates that the circulation of the gas for the amount of time achieved a 6-Log CFU reduction (“negative” result). Conversely, cell growth within the seven-day incubation period indicates at least one viable organism survived the circulation of the gas for the amount of time (“positive” result).

In this example, the seven Bls of FIG. 3 may be in one or more locations in the chamber interior (e.g., with at least some in locations that are relatively difficult to decontaminate in the chamber interior). The Bls may be inoculated with viable spores of Geobacillus stearothermophilus, for example, or another suitable type of thermophile bacteria. The D-value of the Bls may be provided by the manufacturer of the Bls and may be, for example, 0.25 minutes, 0.5 minutes, 1 minutes, 1.5 minutes, 2 minutes, 2.5 minutes, or any other suitable D-value.

Assuming the hypothetical data of FIG. 3 correspond to the same isolator as the hypothetical data of FIG. 2, the dose may be assumed to be 8 mL, in which case the hypothetical data in FIG. 3 primarily serve to help determine the dwell time.

For a dose of 8 mL, the table 300 shows that circulating the gas for 100 seconds results in all but one BI (BI #4) achieving no growth. Because 100 seconds does not achieve no growth for all Bls, 100 seconds is not selected as the dwell time. On the other hand, both 300 seconds and 500 seconds achieve no growth for all seven Bls. Therefore, 300 seconds is the least amount of time (among the amounts of time tested) for achieving no growth for all seven Bls. Accordingly, 300 seconds may be selected as the dwell time, in this example.

It is also worth noting that the hypothetical data of FIG. 3 illustrate that once full saturation is met for the full dwell time, increasing the dose further will not improve CFU reduction. This is observed as the growth-no growth behavior with respect to time is identical for the 8 mL and 10 mL amounts of gas (assuming 8 mL achieves full saturation over the amounts of time, consistent with the hypothetical results of FIG. 2).

While the table 300 only shows one trial/experiment for each combination of dose and dwell time, it is understood that multiple trials/experiments may be conducted. For example, each trial/experiment may be performed in triplicate to help verify accuracy of results. In another example, multiple trials/experiments may be performed with each using Bls of different D-values and/or Bls in different locations.

It is understood that the result of 300 seconds as the dwell time may be unique to this example and may depend on a number of factors, such as the size and geometry of the chamber interior being decontaminated, concentration of the gas, temperature and humidity of the chamber interior, etc. For example, if the chamber interior is the isolator 100 illustrated in FIG. 1, the dwell time may vary between the three different described decontamination cycles. Furthering this example, the Full Cycle Decontamination may have a larger chamber interior volume than the Decontamination Staging Isolator Decontamination Cycle. Accordingly, the dwell time required for the Full Cycle Decontamination may be larger than the dwell time required for the Decontamination Staging Isolator Decontamination Cycle. It may also be the case that the difference in chamber volume interior between the Full Cycle Decontamination and the Filling Isolator Decontamination Cycle is not significant enough to warrant different dwell times. Accordingly, the dwell time required for the Full Cycle Decontamination may be the same as the dwell time required for the Filling Isolator Decontamination Cycle.

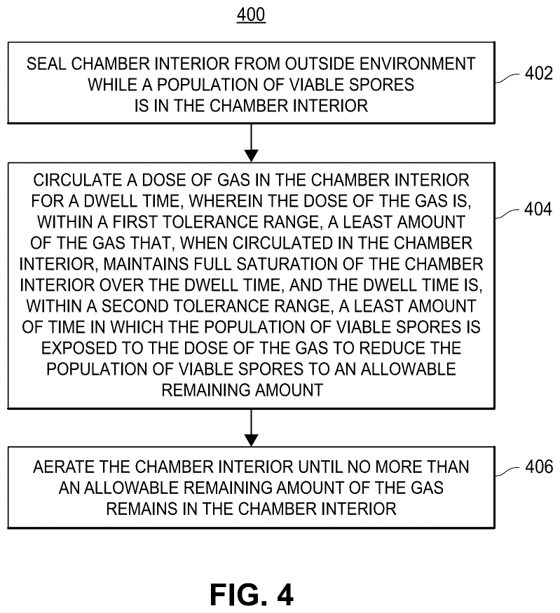

FIG. 4 is a flow diagram depicting an example method 400 of decontaminating a chamber interior using a vapor phase hydrogen peroxide decontamination cycle.

In the depicted method 400, the chamber interior is first sealed from an outside environment while a population of viable spores is in the chamber interior (block 402). The population of viable spores may be included on one or more Bls which may be located in one or more locations in the chamber interior. The population of viable spores may be a population of bacteria, such as a thermophile bacteria (e.g., Geobacillus stearothermophilus). In some embodiments, the population of viable spores includes between 1 and 5 million colony forming units. The “outside environment” may be anywhere outside the chamber interior. For example, if the chamber interior is the filling isolator chamber 104 of FIG. 1, the outside environment may include the DSI chamber 102, a laboratory environment in which the isolator is located, etc. The chamber interior may be considered sealed if the chamber interior is “airtight” with respect to the outside environment (e.g., not allowing for air, gas including hydrogen peroxide, or any other gases to flow between the chamber interior and the outside environment). The volume of the chamber interior may be 50 cubic meters, 25 cubic meters, 10 cubic meters, 5 cubic meters, 1 cubic meter, or some other suitable interior volume. Sealing the chamber interior from the outside environment, as in block 402, may be performed by at least one of one or more processors providing instruction to hardware, or by a human. For example, a human may enter an instruction (e.g., press a virtual button on a graphical user interface generated by one or more processors) to begin a VPHP decontamination process, and in response, one or more processors may trigger one or more actuators that seal the chamber interior.

Next, in the depicted method 400, a dose of gas comprising hydrogen peroxide is circulated in the chamber interior for a dwell time, wherein the dose of gas is, within a first tolerance range, a least amount of gas that, when circulated in the chamber interior, maintains full saturation of the chamber interior over the dwell time, and the dwell time is, within a second tolerance range, a least amount of time in which the population of viable spores is exposed to the dose of the gas to reduce the population of viable spores to an allowable remaining amount (block 404). In some embodiments, prior to circulating the gas in the chamber interior, a vacuum is created in the chamber interior. The first tolerance range and the second tolerance range each may be, for example, no more than 30%, 20%, 15%, 10%, 5%, 3%, 1%, 0.5%, or some other suitable tolerance. The tolerance percentage may be measured with respect to either the determined values (i.e., the determined dose and the determined dwell time) or with respect to theoretically optimal values (i.e., optimal dose and the optimal dwell time). The chamber interior may be a decontamination staging isolator chamber (e.g., the decontamination staging isolator 102 of FIG. 1) and/or a filling isolator chamber (e.g., the filling isolator chamber 104 of FIG. 1) of an isolator (e.g., the isolator 100 of FIG. 1). The gas may be 10% hydrogen peroxide, 20% hydrogen peroxide, 30% hydrogen peroxide, 40% hydrogen peroxide, 50% hydrogen peroxide, 60% hydrogen peroxide, 70% hydrogen peroxide, 80% hydrogen peroxide, or any other suitable concentration of hydrogen peroxide. The dose may be 1 mL, 2 mL, 3 mL, 4 mL, 5 mL, 6 mL, 7 mL, 8 mL, 9 mL, 10 mL, 15 mL, 20 mL, 25 mL, or any other suitable dose of gas. The dwell time may be 10 seconds, 30 seconds, 60 seconds, 100 seconds, 200 seconds, 300 seconds, 400 seconds, 500 seconds, 750 seconds, 1000 seconds, or any other suitable dwell time. The dwell time may be between 250 and 350 seconds, between 300 and 500 seconds, or in any other suitable range. In a more specific example, the gas may be between 45% and 55% concentration hydrogen peroxide, and the dose of the gas may contains between 6 and 10 ml of the gas. In another example, the gas may be between 45% and 55% concentration hydrogen peroxide, and the dose of the gas may contains between 4 and 8 mL. In another example, the gas may be between 25% and 75% concentration hydrogen peroxide. The dwell time may be between 1 and 2 times, 2 and 3 times, 3 and 4 times, 5 and 6 times, 6 and 7 times, 7 and 8 times, 8 and 9 times, or 9 and 10 times a manufacturer-assigned D-value of the population of viable spores, or some other suitable amount of time relative to the assigned D-value. The allowable remaining amount of the population of viable spores may be 100 ppm, 10 ppm, 2 ppm, 1 ppm, or some other suitable amount. Circulating the dose of gas in the chamber interior for the dwell time, as in block 404, may be performed by at least one of one or more processors that control hardware via commands or control signals, or by a human. For example, a human may input the dose and the dwell time (e.g., by entering numbers on a graphical user interface generated by one or more processors), and one or more processors may cause a pump, fan, or other device to release and/or circulate the dose of gas in the chamber interior for the dwell time.

Finally, in the depicted method 400, the chamber is aerated until no more than an allowable remaining amount of the gas remains in the chamber interior (block 406). The allowable remaining amount of the gas may be 100 ppm, 10 ppm, 2 ppm, 1 ppm, or some other allowable amount of the gas. The allowable remaining amount of the gas may be with respect to air. For example, if the allowable remaining amount of the gas is 1 ppm, this would mean for every million parts air in the chamber interior, there is one part the gas. Aerating the chamber, as in block 406, may be performed by at least one of one or more processors providing instruction to hardware, or by a human. For example, a human may input the allowable remaining amount of the gas (e.g., by entering numbers on a graphical user interface generated by one or more processors), and one or more processors may cause a pump or other device to aerate the chamber until no more than an allowable remaining amount of the gas remains in the chamber interior.

Once the method 400 is complete, the chamber interior may be considered decontaminated. For the example of the isolator 100 of FIG. 1, if the method 400 were performed in accordance with Full System Decontamination, the filling isolator chamber 104 and the interior door 110 seals may be considered decontaminated. If the method 400 were performed in accordance with Filling Isolator Decontamination Cycle, the filling isolator chamber 104 of FIG. 1 may be considered decontaminated. If the method 400 were performed in accordance with Decontamination Cycle Isolator Decontamination Cycle, the DSI chamber 102 of FIG. 1 may be considered decontaminated.

The method 400 may be performed entirely by a human operator, in some embodiments. Alternatively, the method 400 may be performed entirely by automation, e.g., by one or more processors (e.g., a CPU and/or GPU) that execute instructions stored on one or more non-transitory, computer-readable storage media (e.g., a volatile memory or a non-volatile memory, a read-only memory, a random-access memory, a flash memory, an electronic erasable program read-only memory, and/or one or more other types of memory). In still other embodiments, the method 400 is performed in part by a human operator, and in part by one or more processors executing instructions.

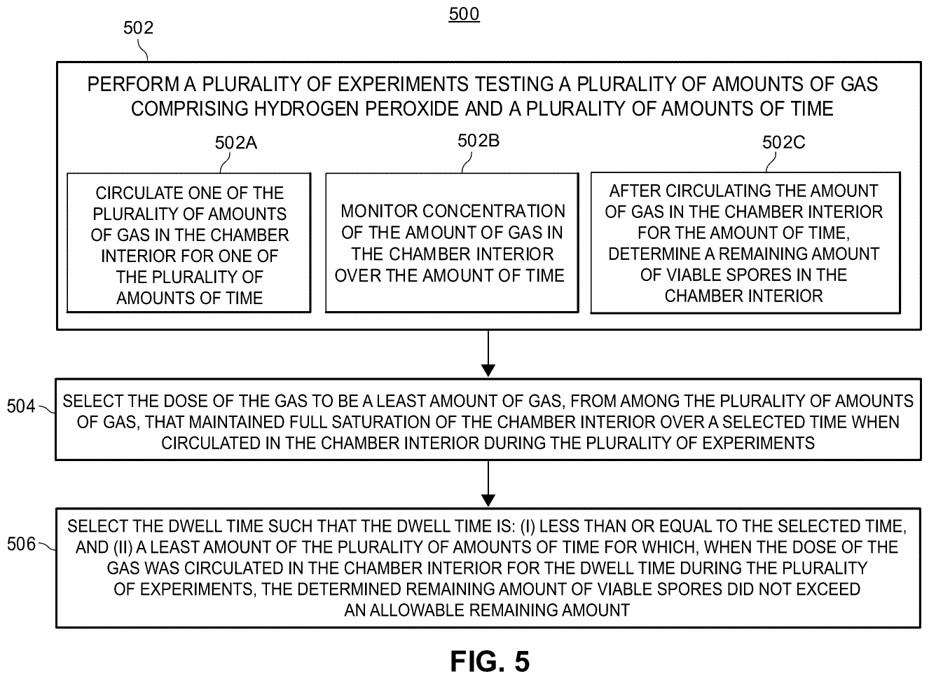

FIG. 5 is a flow diagram depicting an example method 500 of selecting parameters for a decontamination cycle for a chamber interior using a vapor phase hydrogen peroxide decontamination cycle. Specifically, the parameters include at least a dose and a dwell time for a VPHP decontamination cycle.

In the depicted method 500, a plurality of experiments is performed, testing a plurality of amounts of gas comprising hydrogen peroxide and a plurality of amounts of time (block 502). For example, there may be five different amounts of gas tested and five different amounts of time tested, resulting in a total of twenty-five experiments, assuming each experiment is performed once. Alternatively, if each experiment is performed in triplicate, a total of seventy-five experiments may be performed.

For each experiment of the plurality of experiments, an amount of gas (i.e., one of the plurality of amounts of gas) is circulated in the chamber interior for one of the plurality of amounts of time (block 502a). For each experiment of the plurality of experiments, the concentration of the amount of gas in the chamber interior is monitored over the amount of time (block 502b). The concentration of the amount of gas in the chamber interior may be monitored continuously or discretely using a suitable measurement device (e.g., a refractometer). For each experiment of the plurality of experiment, after circulating the amount of gas in the chamber interior for the amount of time, a remaining amount of viable spores in the chamber interior is determined (block 502c). Determining a remaining amount of viable spores in the chamber interior may be accomplished in a number of ways. One example method of a determining the remaining amount of viable spores in the chamber interior includes removing Bls containing the population of viable spores and incubating the Bls in a growth promoting media for seven days. Assuming the Bls were inoculated with a population of viable spores including more than one million CFUs, no growth after seven days indicates that less than 1 ppm of the population of viable spores remains. Cell growth within the seven-day incubation period indicates that more than 1 ppm of the population of viable spores remains.

Next, in the depicted method 500, the dose of the gas is selected to be a least amount of gas, from among the plurality of amounts of gas, that maintained full saturation of the chamber interior over a selected time when circulated in the chamber interior during the plurality of experiments (block 504). Full saturation may be identified by a plateau or “table top” appearance, as illustrated in FIG. 2. In one embodiment, the selected time must be an equal or longer amount of time than the dwell time. In this embodiment the dwell time may not be known yet when determining the selected time, and the selected time may need to be selected to be sufficiently large such that it is equal to or larger than what the dwell time is anticipated to be. For example, the selected time may be selected based on the D-value of the population of viable spores.

Finally, in the depicted method 500, the dwell time is selected such that the dwell time is: (i) less than or equal to the selected time, and (ii) a least amount of the plurality of amounts of time for which, when the dose of the gas was circulated in the chamber interior for the dwell time during the plurality of experiments, the determined remaining amount of viable spores did not exceed an allowable remaining amount (block 506). In one embodiment, the dwell time must be less than or equal to the selected time. If the dwell time is larger than the selected time, it may not be possible to be confident that full saturation of the chamber interior would be maintained over the dwell time. Thus, it may be necessary to repeat at least a portion of the plurality of experiments using a larger selected time (possibly in an iterative manner). The gas may be 10% hydrogen peroxide, 20% hydrogen peroxide, 30% hydrogen peroxide, 40% hydrogen peroxide, 50% hydrogen peroxide, 60% hydrogen peroxide, 70% hydrogen peroxide, 80% hydrogen peroxide, or any other suitable concentration of hydrogen peroxide. The dose may be 1 mL, 2 mL, 3 mL, 4 mL, 5 mL, 6 mL, 7 mL, 8 mL, 9 mL, 10 mL, 15 mL, 20 mL, 25 mL, or any other suitable dose of gas. The dwell time may be 10 seconds, 30 seconds, 60 seconds, 100 seconds, 200 seconds, 300 seconds, 400 seconds, 500 seconds, 750 seconds, 1000 seconds, or any other suitable dwell time. The dwell time may be between 250 and 350 seconds, between 300 and 500 seconds, or in any other suitable range. In a more specific example, the gas may be between 45% and 55% concentration hydrogen peroxide, and the dose of the gas may contain between 6 and 10 ml of the gas. In another example, the gas may be between 45% and 55% concentration hydrogen peroxide, and the dose of the gas may contain between 4 and 8 mL. In another example, the gas may be between 25% and 75% concentration hydrogen peroxide. The dwell time may be between 1 and 2 times, 2 and 3 times, 3 and 4 times, 5 and 6 times, 6 and 7 times, 7 and 8 times, 8 and 9 times, or 9 and 10 times a manufacturer-assigned D-value of the population of viable spores, or some other suitable amount of time relative to the assigned D-value. The allowable remaining amount of the population of viable spores may be 100 ppm, 10 ppm, 2 ppm, 1 ppm, or some other suitable amount.

In some embodiments, the method 500 also includes, after block 506, using the selected dose and dwell time to perform VPHP decontamination (e.g., according to the method 400 depicted in FIG. 4).

The method 500 may be performed entirely by a human operator, in some embodiments. Alternatively, the method 500 may be performed entirely by automation, e.g., by one or more processors (e.g., a CPU and/or GPU) that execute instructions stored on one or more non-transitory, computer-readable storage media (e.g., a volatile memory or a non-volatile memory, a read-only memory, a random-access memory, a flash memory, an electronic erasable program read-only memory, and/or one or more other types of memory). In still other embodiments, the method 500 is performed in part by a human operator, and in part by one or more processors executing instructions. For example, a human may enter an instruction (e.g., press a virtual button on a graphical user interface generated by one or more processors) to begin an experiment, and in response, one or more processors may trigger one or more actuators, pumps, fans, or other devices to perform the experiment.

Some of the figures described herein illustrate example block diagrams having one or more functional components. It will be understood that such block diagrams are for illustrative purposes and the devices described and shown may have additional, fewer, or alternate components than those illustrated. Additionally, in various embodiments, the components (as well as the functionality provided by the respective components) may be associated with or otherwise integrated as part of any suitable components.

Some embodiments of the disclosure relate to a non-transitory computer-readable storage medium having instructions/computer-readable storage medium thereon for performing various computer-implemented operations. The term “instructions/computer-readable storage medium” is used herein to include any medium that is capable of storing or encoding a sequence of instructions or computer codes for performing the operations, methodologies, and techniques described herein. The media and computer code may be those specially designed and constructed for the purposes of the embodiments of the disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as optical disks; and hardware devices that are specially configured to store and execute program code, such as ASICs, programmable logic devices (“PLDs”), and ROM and RAM devices.

Examples of computer code include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel. Another embodiment of the disclosure may be implemented in hardwired circuitry in place of, or in combination with, machine-executable software instructions.

As used herein, the singular terms “a,” “an,” and “the” may include plural referents, unless the context clearly dictates otherwise.

As used herein, the terms “approximately,” “substantially,” “substantial,” “roughly” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, two numerical values can be deemed to be “substantially” the same if a difference between the values is less than or equal to ±10% of an average of the values, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the present disclosure has been described and illustrated with reference to specific embodiments thereof, these descriptions and illustrations do not limit the present disclosure. It should be understood by those skilled in the art that various changes may be made and equivalents may be substituted without departing from the true spirit and scope of the present disclosure as defined by the appended claims. The illustrations are not necessarily drawn to scale. There may be distinctions between the artistic renditions in the present disclosure and the actual apparatus due to manufacturing processes, tolerances and/or other reasons. There may be other embodiments of the present disclosure which are not specifically illustrated. The specification (other than the claims) and drawings are to be regarded as illustrative rather than restrictive. Modifications may be made to adapt a particular situation, material, composition of matter, technique, or process to the objective, spirit and scope of the present disclosure. All such modifications are intended to be within the scope of the claims appended hereto. While the techniques disclosed herein have been described with reference to particular operations performed in a particular order, it will be understood that these operations may be combined, sub-divided, or re-ordered to form an equivalent technique without departing from the teachings of the present disclosure. Accordingly, unless specifically indicated herein, the order and grouping of the operations are not limitations of the present disclosure.

1. A method for decontaminating a chamber interior using a vapor phase hydrogen peroxide decontamination cycle, comprising:
sealing the chamber interior from an outside environment while a population of viable spores is in the chamber interior;
circulating a dose of gas comprising hydrogen peroxide in the chamber interior for a dwell time, wherein:
the dose of the gas is, within a first tolerance range, a least amount of the gas that, when circulated in the chamber interior, maintains full saturation of the chamber interior over the dwell time, and
the dwell time is, within a second tolerance range, a least amount of time in which the population of viable spores is exposed to the dose of the gas to reduce the population of viable spores to an allowable remaining amount; and
after circulating the dose of the gas in the chamber interior for the dwell time, aerating the chamber interior until no more than an allowable remaining amount of the gas remains in the chamber interior,
wherein the first tolerance range and the second tolerance range are each no more than 20%.

2. The method of claim 1, wherein the first tolerance range and the second tolerance range are each no more than 10%.

3. The method of claim 1, wherein the first tolerance range and the second tolerance range are each no more than 5%.

4. The method of claim 1, wherein the first tolerance range is different than the second tolerance range.

5. The method of claim 1, wherein the gas is between 25% and 75% concentration hydrogen peroxide.

6. The method of claim 1, wherein the gas is between 45% and 55% concentration hydrogen peroxide.

7. The method of claim 1, wherein the chamber interior comprises an interior of a chamber of an isolator.

8. The method of claim 7, wherein the vapor phase decontamination cycle is a full system decontamination cycle, and wherein the chamber interior comprises interiors of a decontamination staging isolator chamber and a filling isolator chamber.

9. The method of claim 7, wherein the vapor phase decontamination cycle is a filling isolator decontamination cycle, and wherein the chamber interior comprises an interior of a filling isolator chamber.

10. The method of claim 8, wherein the gas is between 45% and 55% concentration hydrogen peroxide, and wherein the dose of the gas contains between 6 and 10 milliliters of the between 45% and 55% concentration hydrogen peroxide.

11. The method of claim 8, wherein the dwell time is between 300 and 500 seconds.

12. The method of claim 8, wherein the method decontaminates the filling isolator chamber.

13. The method of claim 7, wherein the vapor phase decontamination cycle is a decontamination staging isolator decontamination cycle, and wherein the chamber interior comprises an interior of a decontamination staging isolator chamber.

14. The method of claim 13, wherein the gas is between 45% and 55% concentration hydrogen peroxide, and wherein the dose of the gas is between 4 and 8 milliliters of the between 45% and 55% concentration hydrogen peroxide.

15. The method of claim 13, wherein the dwell time is between 250 and 350 seconds.

16. The method of claim 13, wherein the method decontaminates the decontamination staging isolator.

17. The method of claim 1, wherein a volume of the chamber interior is between 3 and 10 cubic meters.

18. The method of claim 1, wherein a volume of the chamber interior is between 10 and 20 cubic meters.

19. The method of claim 1, wherein the allowable remaining amount of the population of viable spores is 1 part per million of the population of viable spores.

20. The method of claim 1, wherein the allowable remaining amount of the population of viable spores is 2 parts per million of the population of viable spores.

21. The method of claim 1, wherein the allowable remaining amount of the population of viable spores is 10 parts per million of the population of viable spores.

22. The method of claim 1, wherein the dwell time is between 5 and 7 times an assigned D-value of the population of viable spores.

23. The method of claim 22, wherein the dwell time is 6 times the assigned D-value of the population of viable spores.

24. The method of claim 22, wherein the assigned D-value of the population of viable spores is between 0.5 and 2.5 minutes.

25. The method of claim 1, wherein the population of viable spores is a population of bacteria.

26. The method of claim 25, wherein the population of viable spores is a population of thermophile bacteria.

27. The method of claim 26, wherein the population of viable spores is a population of Geobacillus stearothermophilus bacteria.

28. The method of claim 1, wherein the population of viable spores is inoculated on a plurality of biological indicators located in a plurality of locations in the chamber interior.

29. The method of claim 28, wherein each of the plurality of biological indicators includes between 1 and 5 million colony forming units.

30. The method of claim 1, wherein the allowable remaining amount of the gas is 1 part per million of the gas in air in the chamber interior.

31. The method of claim 1, wherein the allowable remaining amount of the gas is 2 parts per million of the gas in air in the chamber interior.

32. The method of claim 1, wherein the allowable remaining amount of the gas is 10 parts per million of the gas in air in the chamber interior.

33. The method of claim 1, further comprising:
removing ambient air from the chamber interior until an allowable remaining amount of the ambient air remains in the chamber interior.

34.-60. (canceled)
